(19) United States
(12) Patent Application Publication
(10) Pub. No.: US 2012/0302890 A1
(43) Pub. Date: Nov. 29, 2012

(54) CRADLE APPARATUS FOR A STEPPER TO HOLD ULTRA-SOUND PROBE

(75) Inventor: David Strong, Carmichaels, PA (US)

(73) Assignee: DAVID STRONG

(21) Appl. No.: 13/116,071

(22) Filed: May 26, 2011

(51) Int. Cl. A61B 8/00

(52) U.S. Cl. 600/459

(57) ABSTRACT

A cradle is connected to a stepper-stabilizer via a plurality of cradle fix holes, wherein said cradle further comprises, a locking knob that is attached to a side of said cradle used for tightening and loosening; a capture knob that is mounted inside said locking knob; a clamp that is connected on top of said cradle used to wrap over and on top of a probe; a probe lock fixer that is attached to said clamp; a cradle base affixed between said cradle and said stepper-stabilizer that allows for movement change in angular rotation of the horizontal axis of said cradle while being connected to said stepper-stabilizer; a plurality of roller-bearings attached between said cradle base and said cradle; and a perpendicular locker attached along a rail of said cradle base used to fix said cradle in a set predetermine angle.

### CROSS REFERENCE TO RELATED APPLICATION

N/A

### FIELD OF THE INVENTION

The invention generally relates to a unique method of holding an ultra-sound probe used to provide brachytherapy.

### BACKGROUND

The concept of insertion of radioactive sources into the prostate for the treatment of prostate cancer has a long history and dates back to the turn of the century. Many patients with prostate cancer were treated by this method in the 1960s and 1970s. At that time the radioactive seeds were placed in the prostate through an open surgical incision in the lower abdomen. Because of the uncertainty of seed placement by this method, this technique was abandoned.

By the late 1980s, technologic and scientific advances in trans-rectal ultrasound imaging had revived interest in seed implantation for the treatment of prostate cancer. The introduction of trans-rectal ultrasound probes made possible real time interactive placement of radioactive seeds into the prostate. The doctor inserts an ultrasound probe into the rectum and attaches it to a stabilizing device which holds the tool for the duration of the treatment. In addition, the development of 3-D simulation software (similar to 3D glasses used for film viewing) allows accurate determination of the dose delivered to the prostate and surrounding structures, bladder, urethra and rectum. These new tools have permitted the refinement of interstitial brachytherapy for prostate cancer and have resulted in a more accurate method of delivering the dose to the prostate gland.

There are many different tools in providing assistance to the physicians when providing radioactive seeds in the patient. There is an ultra-sound machine that uses a stepper/stabilizer to hold the probe. The stepper/stabilizer will be connected to a medical chair. Below will discussed all the component parts of the stepper/stabilizer.

The stepper is light weight state-of-the-art precision stepping device. The stepper's modular design is adaptable to a variety of transrectal transducers. The stepper features a secure centerline detent featuring 90 degree clockwise and counter-clockwise rotation with easy-to-read marking scales from either side.

The cradle is a device that is connected to the stepper. The cradle is a device that holds an ultrasound probe in a fixed position. The cradle is fixed to the stepper; the stepper allows a gentle fixed distance movement of forwards and backwards. Each step taken by the stepper is a precise measured movement. The cradle will move forward with the ultrasound probe depending on the movement of the stepper.

An ultrasound imaging system with an endo-rectal ultrasound probe. The rectal probe must be able to image in transverse section at least. It is advantageous to use a multi-plane probe to visualize any longitudinal plane in order to see the needle path. The frequency of the probe should range between 5 and 8 MHz in order to achieve the necessary spatial resolution as well as the necessary penetration depth. The endo-rectal ultrasound probe is fixed by the cradle that is connected to the stepper.

The precision stepper enables the user to advance and retract an endo-rectal ultrasound probe in the rectum to image the prostate. The endo-rectal probe is inserted into and fixed to the stepper and positioned by moving the stepper/probe combination. Any endo-rectal probe from any producer can be fit to the stepper. Probe can be rotated around the probe's long axis (±45°). The probe locks into the adjustable center position. Movable length of the probe with the stepper: 100 mm. Scale for position recognition. Step width: 2.5 mm or 5 mm selectable as well as free analog movement in and out of the rectum. Additional free analogue movement of the probe to define the exact starting point for stepwise movement: 50 mm. Template is movable (100 mm) in the direction parallel to the ER probe's long axis and can be fixed in any position.

Needle guide grid system for brachytherapy needles: Matrix of 13×13 individual channels (all channels for 18 Gauge needles—standard) 2 nomenclatures (reversible) of the needle channel rows: 1 to 7 (every second row with number, front side of template) or 0 to 12 (every row with number, back side of template) Row spacing: 5 mm in both cases. 2 nomenclatures (reversible) of the needle channel columns: A to G (every second row with letter, front side of template) or A to M (every row with letter, back side of template). Column spacing: 5 mm in both cases. The template is made of a material used for implants. It is thus fully bio-compatible.

Device that supports the stepper is the stabilizer. The stabilizer is connected to the guide rails of an OR table by a specific table holder. The stabilizer and stepper can be moved and positioned manually by the integrated handle and fixed or loosed in its position in space by turning one knob.

However, recently there have been many different problems with the cradle. The problems with the cradle are the ultra-sound probe will not stay in a fixed position, therefore, not allowing proper align of needle grid to the image on the display screen. Another problem with the cradle is that the cradle does not allow for proper 90° degree calibration, thus providing some reference point to medical personnel at a 90° degree point. Another problem with the cradle is that when providing medical treatment for the patient by the medical personnel, all moving parts on the cradle may come undone, therefore, causing the cradle to be disassembled. The disassembled cradle causes delay in providing treatment to patients. Another problem with many cradles are the needle path verification. The needle path verification can be time consuming when the medical personnel have to calibrate the cradle holding the ultra-sound probe to the needle grid. Many cradles require that the medical personnel remove or adjust the ultra-sound probe, this is very time consuming and does not provide efficient medical treatment. Therefore, there is a need in the art to improve the calibration of the ultra-sound probe and the needle path verification. Furthermore, it would be appreciated to one skilled in the art to allow medical personnel to improve patient treatment time, and provide an efficient treatment for medical personnel.

### SUMMARY OF INVENTION

According to one general aspect, a cradle used to hold a probe used to image a patient prior and during medical procedure which a cradle is connected to a stepper-stabilizer via a plurality of cradle fix holes; wherein the cradle further comprises, a locking knob that is attached to a side of the cradle used for tightening and loosening; a capture knob that is mounted inside the locking knob to prevent the locking knob from detaching from the cradle; a clamp that is connected on top of the cradle used to wrap over and on top of a probe; a probe lock fixer that is attached to the clamp that fastens to the probe when the clamp is in a closed position which results in anchoring the probe inside a probe space; a micro-adjuster that is connected to both sides of the cradle; a cradle base affixed between the cradle and the stepper-stabilizer that allows for movement change in angular rotation of the horizontal axis of the cradle while being connected to the stepper-stabilizer; a plurality of roller-bearings attached between the cradle base and the cradle that rotates the cradle in a smooth angular rotation on the horizontal axis; and a perpendicular locker attached along a rail of the cradle base used to fix the cradle in a set predetermine angle.

Further, the cradle used to hold a probe used to image a patient prior and during medical procedure wherein the micro-adjuster is used to allow the cradle to be calibrated to a predetermined angle with or without the probe affixed to cradle.

Further, the cradle used to hold a probe used to image a patient prior and during medical procedure wherein the micro-adjuster is used rotates the cradle a maximum of 10° (+/−) via mechanically altering the cradle railing used to connect the cradle base.

Further, the cradle used to hold a probe used to image a patient prior and during medical procedure wherein the micro-adjuster is altered the perpendicular lock is alter in the same number of angular degrees as the micro-adjuster.

Further, the cradle used to hold a probe used to image a patient prior and during medical procedure wherein the micro-adjuster maybe altered by a screw or a release button for quick calibration.

Further, the cradle used to hold a probe used to image a patient prior and during medical procedure wherein the clamp rotates on a hinge adjoined to the cradle.

Further, the cradle used to hold a probe used to image a patient prior and during medical procedure wherein the clamp is set to lock by tightening the locking knob.

Further, the cradle used to hold a probe used to image a patient prior and during medical procedure wherein the clamp pushes down on the probe via the probe lock fixer.

Further, the cradle used to hold a probe used to image a patient prior and during medical procedure wherein the probe lock fixer is in a concave fashion to allow from maximum surface area of the probe lock fixer to touch the probe thereby resulting in no angular movement of the probe.

Further, the cradle used to hold a probe used to image a patient prior and during medical procedure wherein the probe space is used to place the probe within the cradle.

### DESCRIPTION OF THE DRAWINGS

FIG. 1 is a diagram illustrating the Best® Cradle on a stepper stabilizer.

FIG. 2 is a diagram illustrating the top of the Best® Cradle.

FIG. 3 is a diagram illustrating the left side view of the Best® Cradle.

FIG. 4 is a diagram illustrating the front view of the Best® Cradle.

FIG. 5 is a diagram illustrating the top view of the Best® Cradle.

FIG. 6 is a diagram illustrating the rear view of the Best® Cradle.

FIG. 7 is a diagram illustrating the bottom view of the Best® Cradle.

### DETAILED DESCRIPTION

The invention generally relates to treatment of prostate cancer treatment by using a stepper-stabilizer to image the patient prior to inserting radioactive seeds for dose treatment.

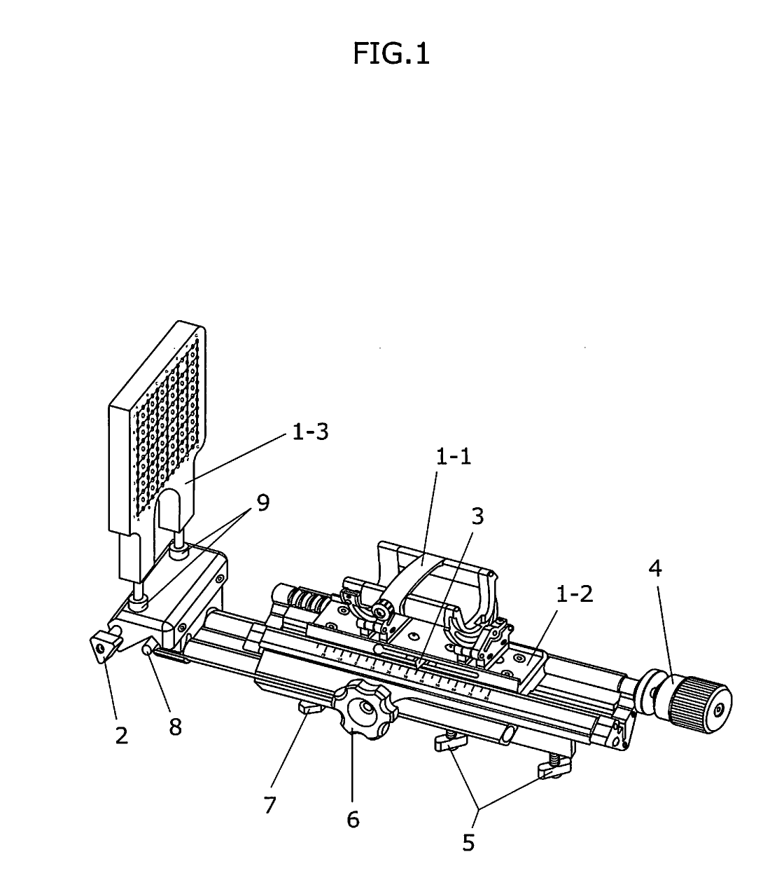

FIG. 1 illustrates a top of view of the Best Cradle 1-1 that is attached to a stepper 1-2. The stepper 1-2 is a piece of hardware that is used to connect to a stabilizer. This overview illustrates the correlation of the Best Cradle to the stepper. Further, attached to the stepper is the needle guide template 1-3. The needle guide template 1-3 is used to provide the location of the needle strand inserted into the human body while being imaged on the patient by a Best NOMOS Sonalis system. The needle guide template 1-3 is fixed on the system to include template locking knob 2. The template locking knob 2 allows a medical assistance to fix the template thus allowing calibration between the needle guide template 1-3 to be precisely aligned with template guide illustrated on the imaging device. However, there are still problems with alignment because of the misalignment of prior cradles and steppers. This can result into placing a radioactive seed in the wrong location as directed by the treatment plan. Next, the stepper has a baseline marker 4. The baseline marker 4 measures the depth of the probe into the human body. This allows medical personnel to insert the probe to a specific depth in the human body to get image front, middle or back part of the prostate. But, the baseline marker 4 can be adjusted by the baseline adjustment knob 6. The baseline adjustment knob 6 allows the medical personnel to calibrate the stepper distance prior to inserting the probe into the human body. Next, the wing nuts 7 are loosen so the mounting screws can slide onto the mounting bracket of the stabilizer. When the wing nuts 7 are tighten and the cradle is attached to the stepper, the stepper knob 8 is used by the medical personnel to move forward and backwards the stepper probe. The major adjustments by medical personnel do not allow for precise calibration of the cradle; however, the Best Cradle was designed to allow for micro-adjustments. The knob may come in many different sizes. Next, the extension knob is used to position the template holder so the stepper maybe fixed. The stepper allows horizontal and vertical needle path adjustments by changing the needle guide template location to match the on-screen grid. When horizontal and vertical adjustments are made; there are still artifacts in the screen display; and the only method to remove these artifacts is for the micro-adjustment developed on the Best Cradle.

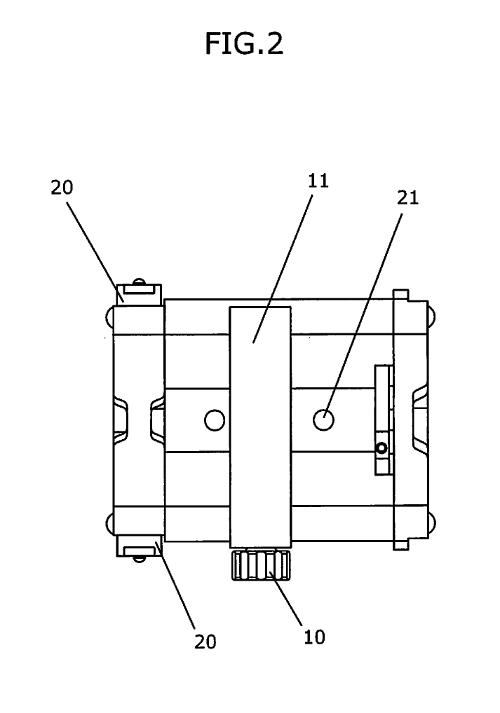

FIG. 2 illustrates the top view of the Best Cradle. The Best Cradle locks a probe by the locking knob 10. The locking knob 10 is tighten by clock-wise motion and loosen by counter-clock wise 10 motion. The clamp 11 is fixed over the probe and is tighten by the locking knob 10. This enclosure allows for the probe to be fixed permanently, since, prior cradles had problems with ultra-sound probes sliding off cradles that results into misalignment or artifacts in the display. Furthermore, the Best Cradle is attached to stepper by screws that attach to the cradle—fix-holes 21.

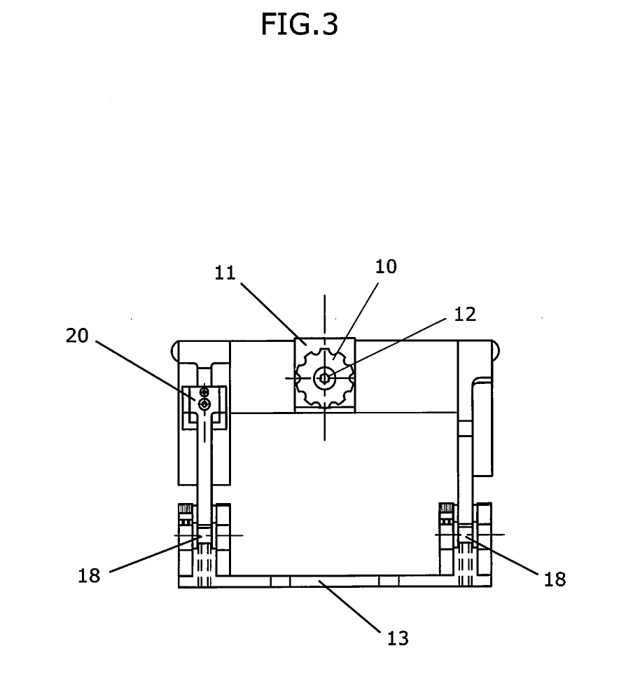

FIG. 3 illustrates a left-side view of the Best Cradle. The locking knob 10 is fixed inside with a capture knob 12. The capture knob 12 prevents the locking knob from falling out of the groove. Next, the micro-adjuster 20 moves the Best Cradle a maximum of 15° (7.5° +/−) degrees. The micro-adjuster 20 is used after the cradle base 13 has been locked in 90 degree perpendicular plane with or without the probe. The cradle base 13 can rotate the Best Cradle for a maximum of 180° degrees (+/−90°) by roller ball-bearings 18. The added benefit of the micro-adjuster 20 is to allow the medical personnel to change the beam plane slightly to correct calibration error. Specifically, some system probes are potted incorrectly with crystal alignment; therefore, the micro-adjuster allows for these manufacturing defects to be corrected by moving the probe within the cradle from the center detent.

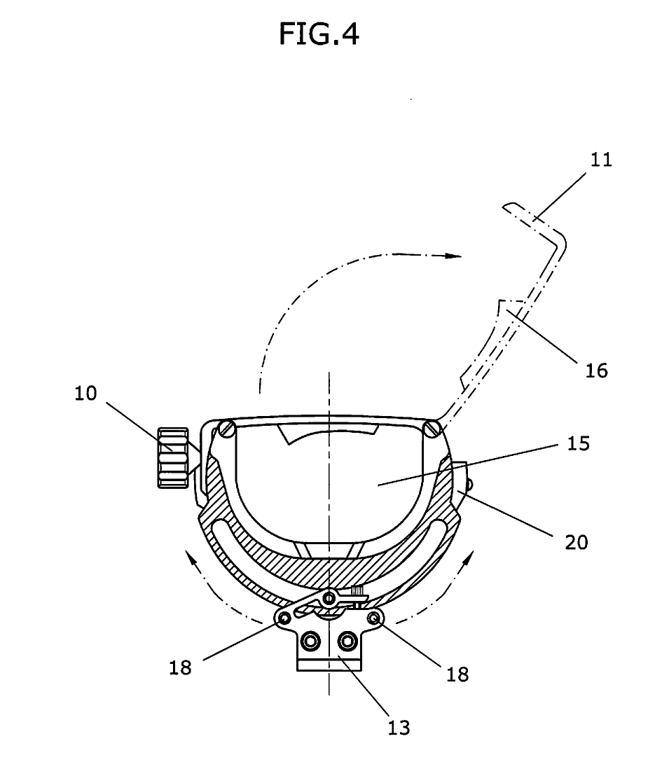

FIG. 4 illustrates a front-view of the Best Cradle. The clamp 11 can open and close over the probe. The probe is placed into probe space 15 and enclosed by the clamp 11. The clamp 11 has a probe-lock-fixer 16 that is attached to the clamp 11. This is used to hold the probe in place. Prior cradles would hold the probe; however, during medical procedures, the probes would rotate within the clamp 1-2 degrees, which affects the imaging quality during the procedure. Therefore, probe-lock-fixer 16 was designed to hold the probe in a fixed position, without causing rotational plain movement. Next, the cradle-base 13 can move in either direction up. Allowing the cradle-base 13 to move in a 180 degree fashion allows for medical personnel to adjust image quality displayed on a computer screen. The cradle-base 13 moves within the cradle by a series of roller ball-bearings 18. The roller ball-bearings 18 allow for smooth sliding of the probe in a fixed position. By rotating the cradle-base 13, the medical personnel is rotating only the degrees of the probe and adjusting the horizontal or vertical lines.

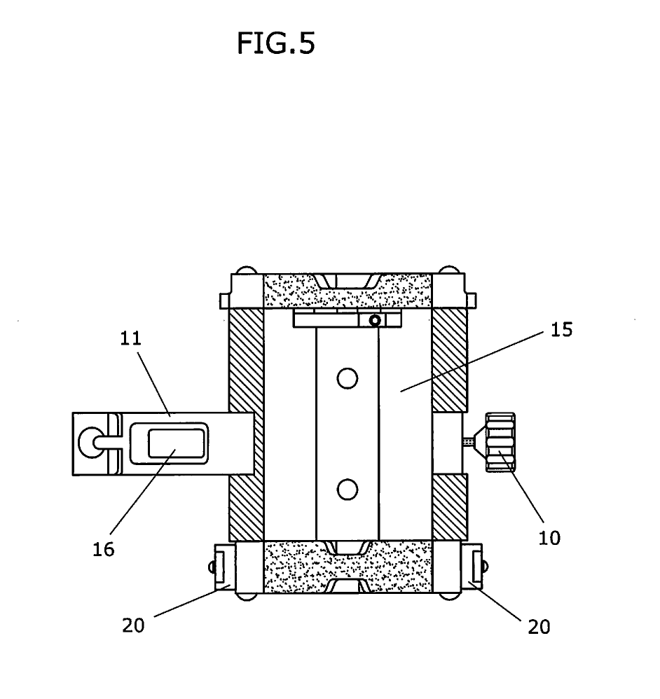

FIG. 5 illustrates a top-view of the Best Cradle with clamp 11 open to show the probe-space 15. The probe is placed into the probe-space and closed. The Best Clamp is first attached to the stepper, and then the probe is attached to the Best Cradle. Furthermore, the claim 11 has a concave enclosure 25 that fixes itself into the lock knob 10. The locking knob has a convex ending 25 that allows for self-tensioning for the clamp and provides the right amount pressure to hold the probe in place.

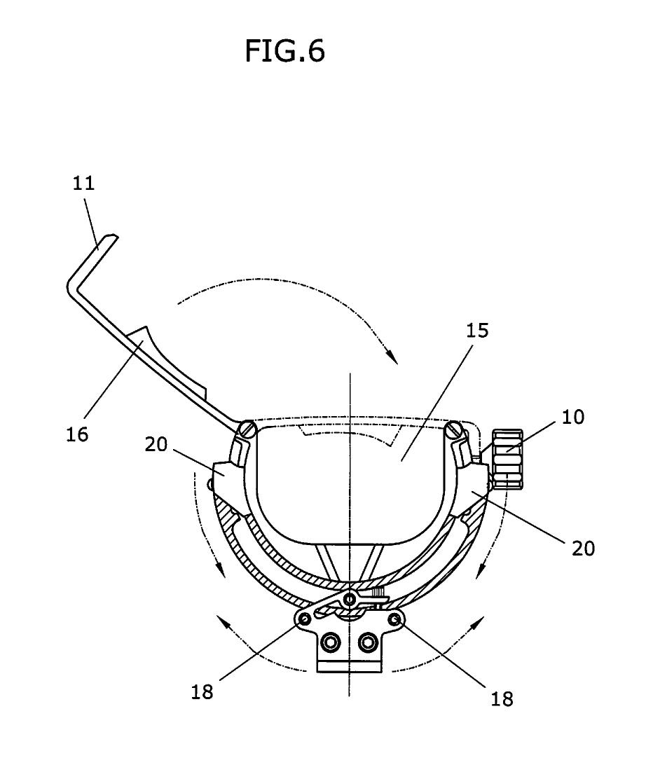

FIG. 6 illustrates a right-side rear view of the Best Cradle with clamp 11 open. The probe-lock-fixer 16 is designed in a concave fashion. The design allows for maximum surface area to be held by the probe-lock-fixer 16 against the probe. The micro-adjuster 20, after being loosened by a nut-screw, will also move up and down as well. The base

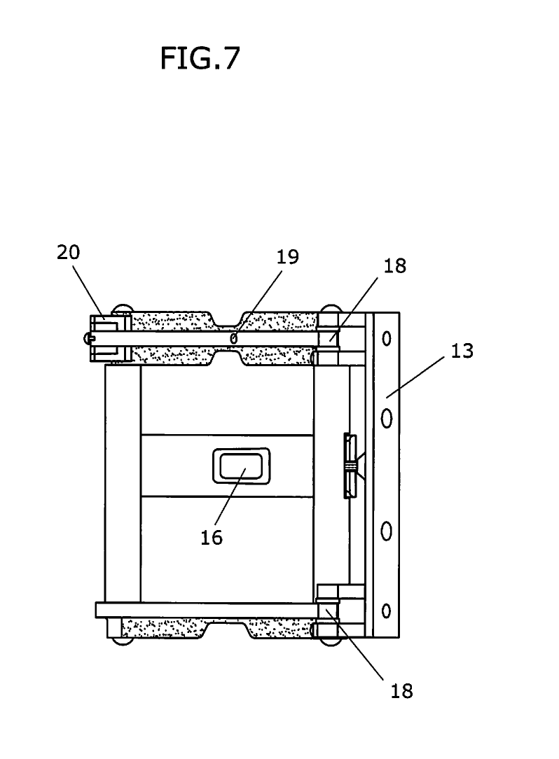

FIG. 7 illustrates a bottom-view of the Best Cradle. The Best Cradle shows the roller ball-bearings 18. The roller ball-bearings 18 are located on both sides of the Best Cradle. As the roller ball-bearing 18 moves from one side to another side, the cradle-base 13 slides over the perpendicular locker 19. The perpendicular locker is used to fix the cradle base 13 in a 90 degree position. Prior art cradles do not have this feature and the medical personnel have to determine the angle prior to inserting the probe into the human body. However, there may be misalignment issue with perpendicular locker 19, thus, the micro-adjuster 20 allows for a medical personnel to readjust the perpendicular locker 19 to the exact 90 degrees. This is very important since this will allow the medical personnel to expedite the needle grid verification.

1. A cradle used to hold a probe used to image a patient prior and during medical procedure which comprises:
a cradle is connected to a stepper-stabilizer via a plurality of cradle fix holes;
wherein said cradle further comprises,
a locking knob that is attached to a side of said cradle used for tightening and loosening;
a capture knob that is mounted inside said locking knob to prevent said locking knob from detaching from said cradle;
a clamp that is connected on top of said cradle used to wrap over and on top of a probe;
a probe lock fixer that is attached to said clamp that fastens to said probe when said clamp is in a closed position which results in anchoring said probe inside a probe space;
a micro-adjuster that is connected to both sides of the said cradle;
a cradle base affixed between said cradle and said stepper-stabilizer that allows for movement change in angular rotation of the horizontal axis of said cradle while being connected to said stepper-stabilizer;
a plurality of roller-bearings attached between said cradle base and said cradle that rotates said cradle in a smooth angular rotation on the horizontal axis; and
a perpendicular locker attached along a rail of said cradle base used to fix said cradle in a set predetermine angle.

2. The cradle used to hold a probe used to image a patient prior and during medical procedure according to claim 1, wherein said micro-adjuster is used to allow said cradle to be calibrated to a predetermined angle with or without said probe affixed to cradle.

3. The cradle used to hold a probe used to image a patient prior and during medical procedure according to claim 2, wherein said micro-adjuster is used rotates said cradle a maximum of 10° (+/−) via mechanically altering the cradle railing used to connect said cradle base.

4. The cradle used to hold a probe used to image a patient prior and during medical procedure according to claim 2, wherein said micro-adjuster is altered said perpendicular lock is alter in the same number of angular degrees as said micro-adjuster.

5. The cradle used to hold a probe used to image a patient prior and during medical procedure according to claim 2, wherein said micro-adjuster maybe altered by a screw or a release button for quick calibration.

6. The cradle used to hold a probe used to image a patient prior and during medical procedure according to claim 1, wherein said clamp rotates on a hinge adjoined to said cradle.

7. The cradle used to hold a probe used to image a patient prior and during medical procedure according to claim 1, wherein said clamp is set to lock by tightening said locking knob.

8. The cradle used to hold a probe used to image a patient prior and during medical procedure according to claim 1, wherein said clamp pushes down on said probe via said probe lock fixer.

9. The cradle used to hold a probe used to image a patient prior and during medical procedure according to claim 1, wherein said probe lock fixer is in a concave fashion to allow from maximum surface area of the said probe lock fixer to touch said probe thereby resulting in no angular movement of said probe.

10. The cradle used to hold a probe used to image a patient prior and during medical procedure according to claim 1, wherein said probe space is used to place said probe within said cradle.
